(19) United States
(12) Patent Application Publication
(10) Pub. No.: US 2011/0262487 A1
(43) Pub. Date: Oct. 27, 2011

(54) De Novo Anembryonic Trophoblast Vesicles and Methods of Making and Using Them

(75) Inventor: Jared Robins, Sharon, MA (US)

(73) Assignee: WOMEN & INFANTS HOSPITAL, Providence, RI (US)

(21) Appl. No.: 12/934,794

(22) PCT Filed: Mar. 26, 2009
(86) PCT No.: PCT/US2009/001897

§ 371 Date: May 10, 2011

Related U.S. Application Data
(60) Provisional application No. 61/070,822, filed on Mar. 26, 2008.

(51) Int. Cl. A61M 31/00; C12N 5/00; A61K 31/12

(52) U.S. Cl. 424/400; 424/93.7; 435/395

(57) ABSTRACT

De novo, anembryonic trophoblast vesicles and methods of making and using them are provided.

### FIELD OF THE INVENTION

The invention is in the field of biotechnology. More specifically the invention is directed to the creation of anembryonic trophoblast vesicles.

### BACKGROUND OF THE INVENTION

From the moment of conception, the human embryo undergoes rapid proliferation and differentiation. By six days postconception, the embryo forms a spherical, fluid-filled blastocyst containing multiple cell lines. The trophoblast cells, destined to become the placenta and embryonic membranes, line the outer rim of the sphere forming a vesicle. The formation of this structure is critical for implantation, normal placental invasion, and embryonic development. However, the mechanism by which the blastocyst forms is unclear. Current research studying trophoblast differentiation relies on monolayer cell culture and tissue explants. Neither of these models is capable of studying the complex cell-to-cell interaction and early differentiation required to form a trophoblast vesicle.

Mammalian embryonic development occurs through rapid cellular division and differentiation. The development occurs within a sphere confined by the zona pellucida, a glycoprotein shell surrounding the embryo. On post-conception day number 3, the pre-embryo contains approximately 8-16 pluripotent blastomeres (depending on species) filling the sphere. Tight junctions begin to form among the blastomeres distorting their shape resulting in a “mulberry”-shaped morula. The tight junctions restrict paracellular passage of ions and small molecules creating a distinct fluid component.^{20, 21} The cells begin to express cell membrane bound sodium-potassium ATPase on their inner surfaces that begin to increase the oncotic pressure in the blastocyst; this results in an increase of fluid in the center of the blastocysts.²² As this fluid accumulates the cells are moved to the outer rim creating a hollow sphere. Two separate cell lines are now identifiable, the trophoblast cells that will develop into to the placenta and fetal membranes and the inner cell mass that will develop into the embryo. Because the trophoblast cells form a hollow sphere they are referred to as trophoblast vesicles.

The human placenta is a highly invasive endocrine organ formed from a progenitor cell, termed the cytotrophoblast, that can be identified in the embryo as early as six days postconception.¹ The cytotrophoblast cell can differentiate along one of two distinct pathways to become either an extravillous or a villous trophoblast cell (see FIG. 1).² The extravillous trophoblast cell is a highly migratory cell type that invades the uterus and maternal vasculature anchoring the embryo and forming the maternal-fetal interface. In contrast, the villous trophoblast cells encompass the embryo and are critical for the synthesis of hormones and growth factors.³ These villous cells make up the majority of the placental mass.

The processes of proliferation, differentiation, and invasion are highly coordinated to establish and maintain a normal pregnancy. It has been postulated that disruptions in the processes lead to pregnancy-related diseases such as recurrent spontaneous abortion, preeclampsia and intrauterine growth restriction.^{4, 5} However, the molecular mechanisms that guide these processes, the overall focus of the principal investigator's research, are not known.

A wealth of information about placental cell differentiation has been gained from traditional two-dimensional monolayer culture. Gene profiling techniques have identified important target genes for studying the pathways. The role of growth factors, such as IGF-1 and TGF-β has been demonstrated and the importance of novel genes such as syncytin, an envelope gene of the recently identified human endogenous retrovirus HERV-W, has been discovered.⁶⁻⁸ Culture techniques have been refined by optimizing the media and culture environment; for example, low oxygen culture promotes the differentiation of cells in the extravillous trophoblast cell pathway.⁹

Despite the important information gained from traditional two-dimensional cell culture, this technique does not allow the study of cell-to-cell interactions. Furthermore, monolayer culture does not allow the study of the gene programs that are required to form complex cellular structures such as trophoblast vesicles and placenta. For these reasons, biologists have attempted to bioengineer three-dimensional cell-culture systems. Many of these systems utilize an extra-cellular matrix, such as fibronectin or alginate, to maintain the three-dimensional shape of the culture.¹⁰⁻¹² Cells are imbedded into the matrix and then cultured. It has been well described that different matrices may have significantly different effects on promoting gene programs and it is critical to experiment with multiple matrices to establish an appropriate in vitro model.^{13, 14}

Synthetic and natural scaffolds have also been used as temporary substrates for native extracellular matrix. This technique is particularly useful for bioengineering micro-tissues that form matrices such as osteoblasts.¹⁵ As with using extracellular matrix components, materials used in scaffold based tissue engineering can affect gene programming. Therefore, it is critical to utilize multiple models. Furthermore, if the bioengineered tissue is ultimately to be transplanted, the scaffold material must be non-antigenic, non-mutagenic, and non-carcinogenic.

Early placental differentiation is unique because it forms in the absence of extra-cellular matrix. As mentioned earlier, the trophoblast cells are the first cells to differentiate in the pre-embryo, approximately 5 to 6 days post-conception. This differentiation is occurring as the pre-embryo is coursing through the fallopian tube without contact with the tubal epithelium. Therefore, three-dimensional culture of this tissue type would be most ideal without the use of any extracellular matrix. Hanging drop culture, one of the oldest cell culture techniques, promotes three-dimensional spheroid formation without extracellular matrix. A small number of cells (100-600) are suspended in a small amount of media and dropped onto an inverted lid of a sterile dish. The lid is then placed on the dish to form a “hanging drop”.¹⁶ The cells cluster, by gravity, to the bottom of the drop and cell-to-cell interactions result in the formation of a spheroid. Hanging drop has been used extensively to culture embryonic stem cells. The stem cells remain undifferentiated in monolayer; in hanging drop culture, the cells begin to differentiate and form embryonic structures. This technique has also been used to culture mice embryos and demonstrate that the cells can be pushed further along the developmental pathway. However, the hanging drop method has several drawbacks. A small number of cells must be used to maintain the drop and the time in culture is limited as the media cannot be changed. Furthermore, the cells cannot be manipulated and remain in the three-dimensional support.

### SUMMARY OF THE INVENTION

In one aspect, the invention comprises functional, de novo, anembryonic trophoblast vesicles. The trophoblast vesicles of the invention are made from trophoblast cells using a cell aggregation device composed of non-adhesive hydrogels containing a plurality of cylindrical recesses; the hydrogels can be cast from molds designed using computer-assisted design rapid prototyping as is fully described in PCT Patent Application PCT/US2007/002050 (PCT Publication No. WO2007/087402), which is incorporated by reference herein in its entirety. This is an improvement over the known method of matrix-free self-assembly known as “hanging drop”.¹⁷⁻¹⁹

Briefly, wax micro-molds are designed using computer assisted design software and produced using a rapid prototyping machine. Molds contain arrays of cylindrical pegs with hemispherical tops, sitting on a rectangular box used to create a cell-seeding chamber (FIG. 2 left side). A hydrogel, for example, polyacrylamide, or agarose is then gelled around the micro-mold; on removal the gel contains an array of recesses complementary to the pegs on the mold (FIG. 2 right side) and forms the cell aggregation device in which cells can aggregate without adherence to the hydrogel substrate. As described in WO2007/097402, the aggregation device has a plurality of cell-repellant compartments recessed into the uppermost surface. Each compartment is composed of an upper cell suspension seeding chamber having an open uppermost portion and a bottom portion, and one or more than one, lower cell aggregation recesses connected at the top to the bottom of the upper cell suspension seeding chamber by a port. The upper cell suspension seeding chambers are formed and positioned to funnel the cells into the lower cell aggregation recesses through gravitational force. The aggregation recesses are formed and positioned to promote cellular aggregation by coalescing cells into a finite region of minimum gravitational energy, increasing intercellular contact and minimizing or preventing cell adherence to the substrate. For agarose or polyacrylamide hydrogels, a depth of between about 1000 to about 2000 μm is preferred for the seeding chambers and between about 500 to about 1000 μm is preferred for the aggregations recesses. The width (horizontal cross-sectional shortest length) of the cell seeding chambers should be at least 2 mm and the aggregations chambers should be between 20 and 5000 μm preferably from about 200 to about 600 μm. A cell suspension seeded into the chamber will settle into the recesses and form a spheroid of uniform size in the bottom of each recess (see FIG. 3). The size and shape of the recess can be altered and these changes are reflected in the properties of the spheroids. The aggregation device then is placed in a sterile dish containing media and the cells are allowed to aggregate into spheroids.

The trophoblast cells seeded into the device form three-dimensional spheroids with an acellular center resembling trophoblast vesicles. Because the agarose substrate is porous, nutrients move across the agarose to support the cells. Therefore, cell culture can be performed for extended periods. The spheroids can easily be removed, manipulated and re-plated into the device. This technique is ideal for bioengineering placental tissue. The experiments outlined herein employ this non-adhesive, micro-mold cell aggregation device to form de novo trophoblast vesicles, the earliest stage of placental development.

The formation of this unique trophoblastic structure is not predicted by the current literature on cellular self-assembly. Data disclosed herein suggest that these vesicles form a functional micro-tissue. Therefore, culturing trophoblast cells in a nonadhesive micro-mold bioreactor, such as the one referenced above, can create de novo functional trophoblast vesicles. The development of these vesicles will significantly improve the ability of scientists to study early embryonic events such as implantation and placental formation leading to a better understanding of diseases including infertility and preeclampsia. Furthermore, the creation of de novo functional trophoblast vesicles can be used by scientists to improve the techniques for genetic manipulation of animals, such as mouse knockout models, and dramatically improve their efficiency.

In another aspect the invention comprises a method of making de novo functional anembryonic trophoblast vesicles. In the method, trophoblast cells are seeded into a non-adhesive cell aggregation device, incubated and allowed to form three-dimensional spheroids with an acellular center. These spheroidal trophoblast vesicles may then be injected with inner cell masses or embryonic stem cells from the same or from a different organism and implanted in a receptive endometrium.

Currently, trophoblast vesicles are anembryonic blastocysts traditionally made by microsurgical removal of the inner cell mass. When transferred to a receptive endometrium, trophoblast vesicles will implant and begin development. In the absence of a viable inner cell mass, this development will arrest. However, the inner cell mass does not need to be from the same blastocyst as the trophoblast. Rossant and colleagues removed the inner cell mass from mouse blastocysts using microsurgical techniques. The blastocyst vesicle reformed a hollow sphere. Inner cell masses from different mice were injected into the vesicle and the newly reconstructed blastocysts were transferred into prepared mouse surrogates. This procedure resulted in the birth of healthy pups of the strain identical to the inner cell mass.^{23, 24} This technique can also be used to for interspecific cloning. Polzin et al injected caprine inner cell masses into ovine vesicles created from ovine blastocysts having their inner cell masses removed and transferred the resultant embryos to ovine surrogates.²⁵ The ewes birthed thirteen young (59% pregnancy rate) of whom ten were caprine, 1 was ovine and 2 were caprine-ovine chimeras. These models are powerful tools to study maternal-fetal interactions during pregnancy, particularly in regard to maternal tolerance and maternal-fetal incompatibility. They have also been employed as a mechanism to rescue endangered species using cloning technology.²⁵ A major obstacle to the development of these technologies is the difficulty in preparing the trophoblast vesicles, using microsurgery, without damaging their morphology. The bioengineering of de novo trophoblast vesicles as described herein eliminates this step. This technique could have significant impact in creating genetically manipulated animals such as knockout mice. By injecting manipulated embryonic stem cells into the de novo trophoblast vesicles of the invention, genetically altered mouse models could be created with 100% somatic cell effect and no affect on the invading placenta.

Embryonic stem cells derived from the blastocyst will self-sort into their tissue of origin. Investigators labeled trophoblast and epiblast cells with green fluorescent protein, injected into the center of the blastocyst and tracked the cells until they incorporated into the blastocysts.^{26, 27} Trophoblast cells contributed exclusively to the trophectoderm while cells derived from the inner cell mass contributed exclusively to the embryo. This phenomenon has been exploited for use in gene therapy and transgenetics²⁸. These therapies are limited by the preparation of both embryonic stem cells and trophoblast vesicles. The bioengineering of the de novo trophoblast vesicles of the invention as described herein has the potential to vastly improve the studies with these techniques and advance this science.

### DESCRIPTION OF THE DRAWINGS

FIG. 1 is a schematic representation of the trophoblast differentiation pathway.

FIG. 2 is a photographic reproduction of the wax micro-mold designed using computer assisted design and rapid prototyping. Molds contain arrays of cylindrical pegs sitting on a cell-seeding chamber. Agarose is gelled around the micro-mold. On removal, the gel contains an array of recesses complementary to the pegs on the mold.

FIG. 3 are photographic reproductions of the trophoblast cell spheroids cultured in non-adhesive, round-bottomed hydrogels cell aggregation devices (bottom panel) and the spheroids removed from the cell aggregation device showing the spheroids remain intact and are manipulatable in culture.

FIG. 4 are photographic reproductions of the trophoblast cell spheroids fixed, sectioned and stained with H & E. A consistent sized rim 12.3μ (+/− 1μ) in the left panel. The hollow structure is confirmed by confocal microscopy through the edge (center panel) and center (right panel) of the spheroid.

FIG. 5 is a photomicrograph of the spheroids in long-term culture. Spheroids labeled with CFDMA demonstrated viability for at least 20 days in culture.

FIG. 6 is a photograph of TCL spheroids placed in cell culture treated dishes (left panel) and on a non-adhesive surface (right panel). The left panel spheroids rapidly adhere to the plate and begin proliferating into a monolayer. The right panel spheroids rapidly fuse together, forming large cellular complexes.

FIG. 7 is a photomicrographic reproduction of the rim of a vesicle at 4400×.

FIG. 8 is a photomicrographic reproduction of the rim at 36000×.

FIG. 9 illustrates the pressure device employed in Example 4 to measure the pressure inside a trophoblast vesicle.

FIG. 10 (a)-(e) are photographic reproduction showing the results of the experiment carried out in Example 10.

FIG. 11 is a schematic representation of an alternative technique to indirectly calculate vesicle pressure.

FIG. 12 is a schematic representation of the system for measurements of pressure inside trophoblast vesicles.

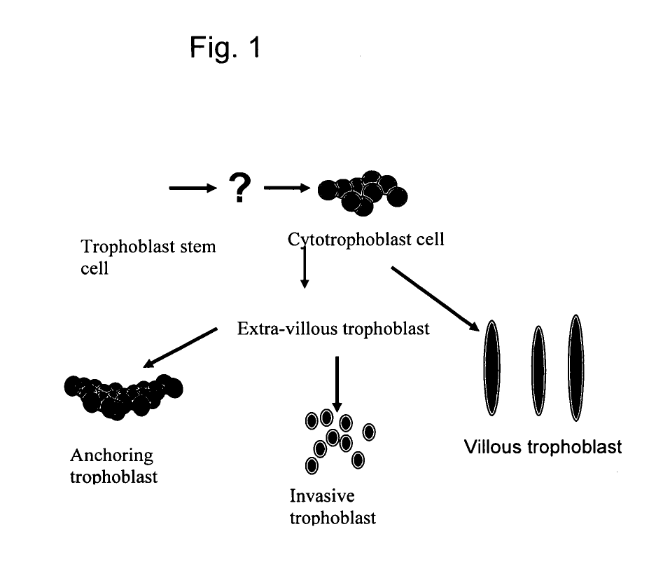

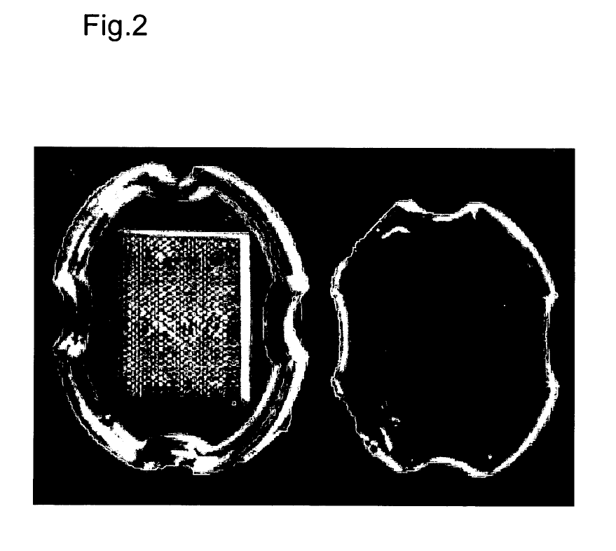

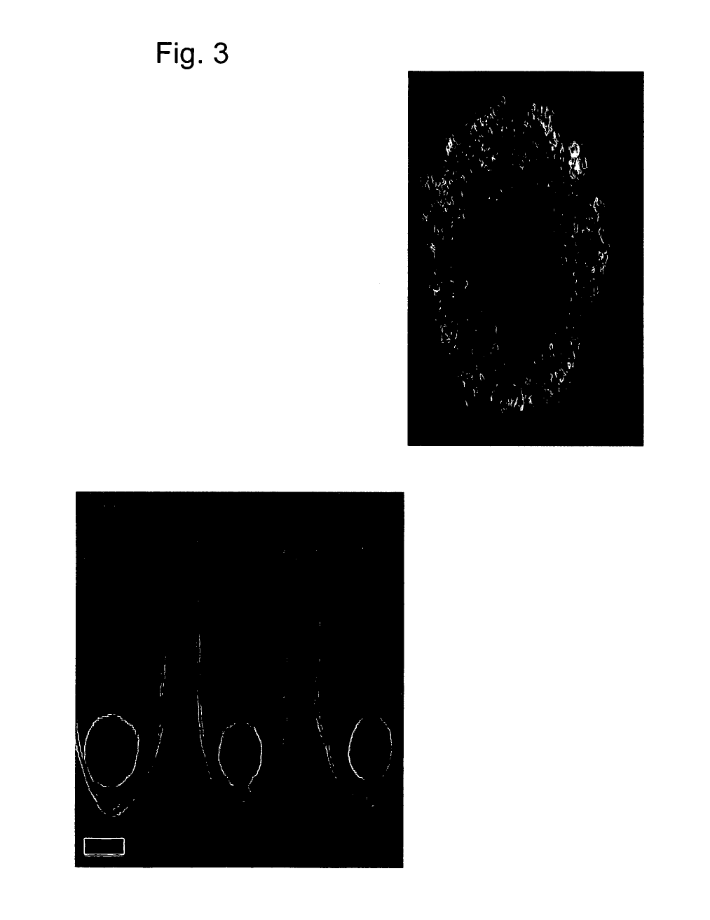

### DETAILED DESCRIPTION

In the following examples, trophoblast cells form de novo anembryonic trophoblast vesicles when placed into a non-adhesive micro-mold cell aggregation device or bioreactor. In this context de novo means from non-aggregated trophoblast cells, in contrast to trophoblast vesicles known in the art, which are created by microsurgical removal of the inner cell mass from blastocysts. The de novo trophoblast vesicles of the invention are morphologically similar to naturally occurring trophoblast vesicles. The cells within the vesicle are metabolically active and form tight-junctions. Further they attach and proliferate across a tissue culture treated dish. These actions indicate that the trophoblast vesicles are biologically functional tissue. This is demonstrated in the following examples.

### Example 1

### Trophoblast Cells Form Trophoblast Vesicles in a Nonadhesive Micro-Mold Bioreactor

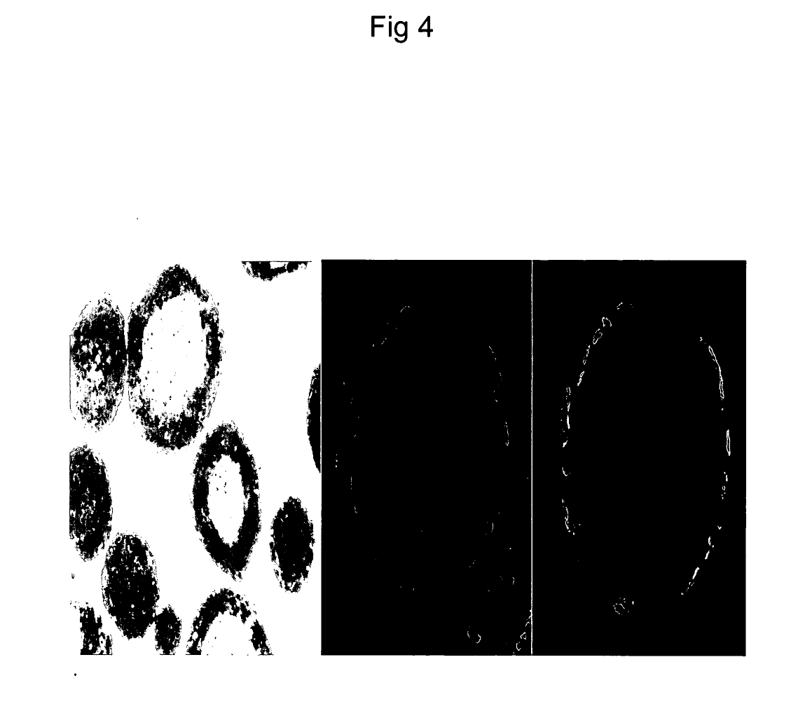

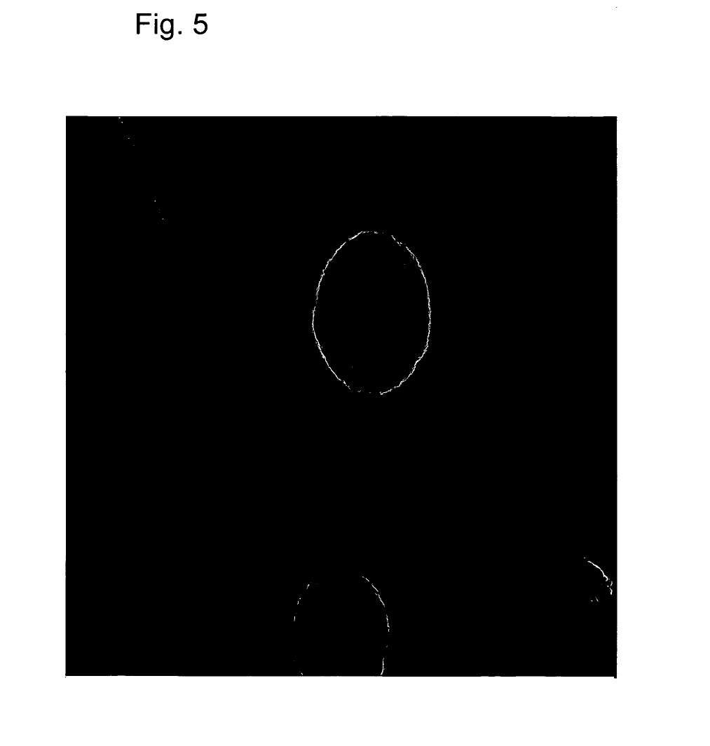

Human immortalized trophoblast cells were seeded into nonadhesive micro-mold bioreactors containing 822 cylindrical recesses 200 μm in diameter. Each recess was seeded with 800,000 cells. The cells settled into the wells within hours of seeding. Within three days, the cells formed uniform sized spheroids approximately 150 um in diameter (see FIG. 4) Determination of the three-dimensional spheroid morphology and was performed on days 7 and 10 after seeding. The spheroids were removed from the molds, fixed and sectioned. Surprisingly, the spheroids contained a hollow center with a cellular rim 12.3 μm (±1 μm) in thickness (see FIG. 4). The morphology did not change significantly between days 7 and 10; the thickness of the rim and the diameter of the center remained constant. The consistent hollow shape of the sphere was confirmed in live cells with confocal imaging after DAPI staining (see FIGS. 4-6). This hollow configuration is not predicted by previous models of spheroid formation and has never been previously reported.

### Example 2

### Trophoblast Cells in Vesicles are Highly Active and Form Tight-Junctions

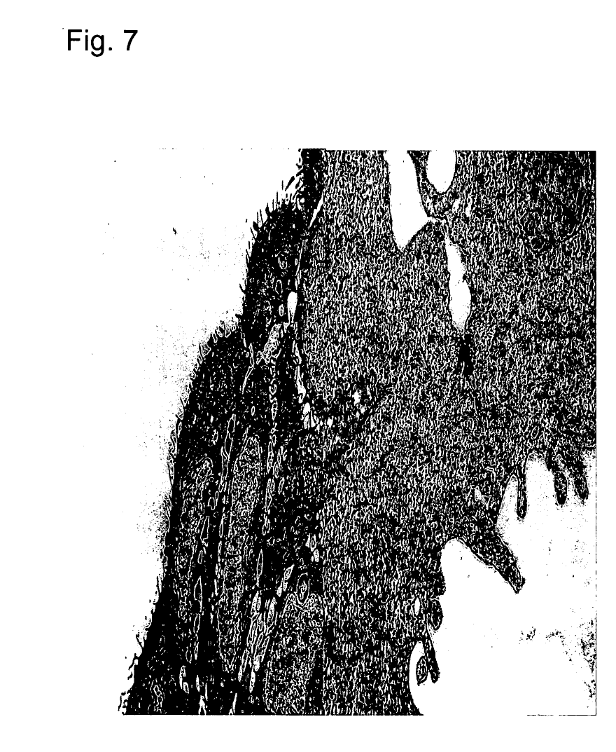

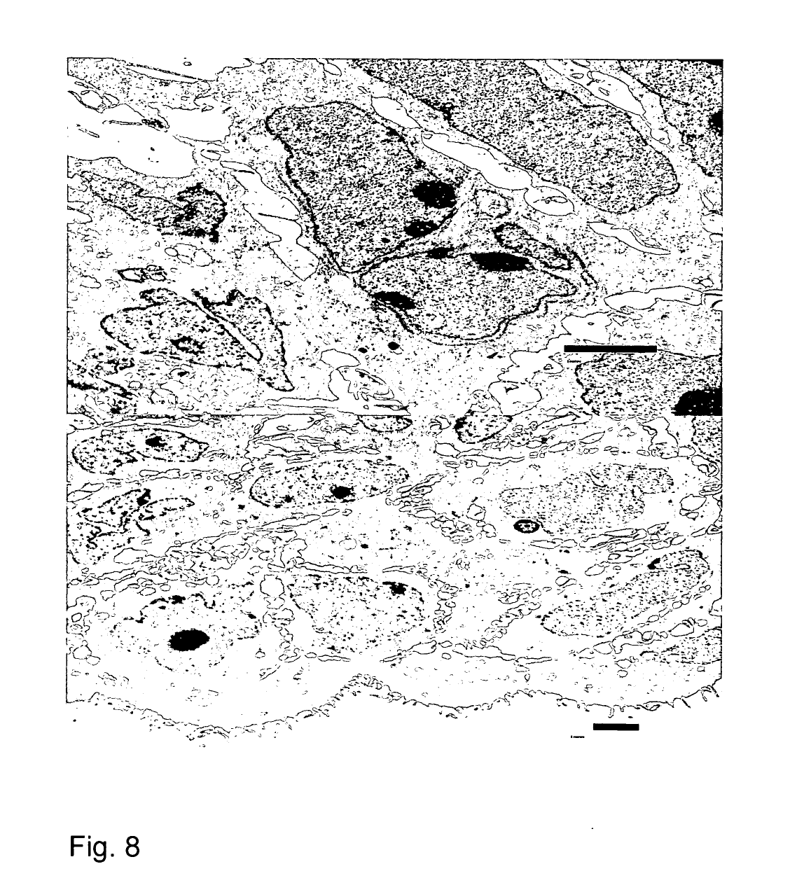

Electron microscopy was performed to further demonstrate cellular morphology and examine cell-to-cell interactions. Close inspection of individual cells at high power demonstrated that the cytoplasm was replete with cellular organelles including large numbers of mitochondria, endoplasmic reticulum and golgi. This confirms that the cells are alive and, more importantly, metabolically active. Mitotic bodies can be identified illustrating that the cells in the rim are proliferating. Apoptotic bodies can be also identified throughout the cellular rim adjacent to mitotic cells. Numerous tight-junctions were identified among the viable cells supporting the notion that cell-to-cell communication is occurring (see FIGS. 7-8). This formation of tight junctions is a hallmark of in vivo trophoblast vesicle formation.

### Example 3

### Trophoblast Vesicles are Highly Adherent

Spheroids removed from the molds adhere to the cell culture dishes within two hours. These cells will proliferate across the surface of the plate and form confluent monolayers. This proliferative action is morphologically similar to a blastocyst attached to a cell culture dish. Upon reseeding into the aggregation device, the cells in monolayer will re-form spheroids. There are no obvious morphologic differences among the spheroids formed by multiple reseedings. These data indicate that the trophoblast cells are forming biologically active vesicles. These de novo vesicles may be employed in implantation, using various known in vitro and in vivo models of tissue function and implantation.

### Example 4

### Measurement of Pressure

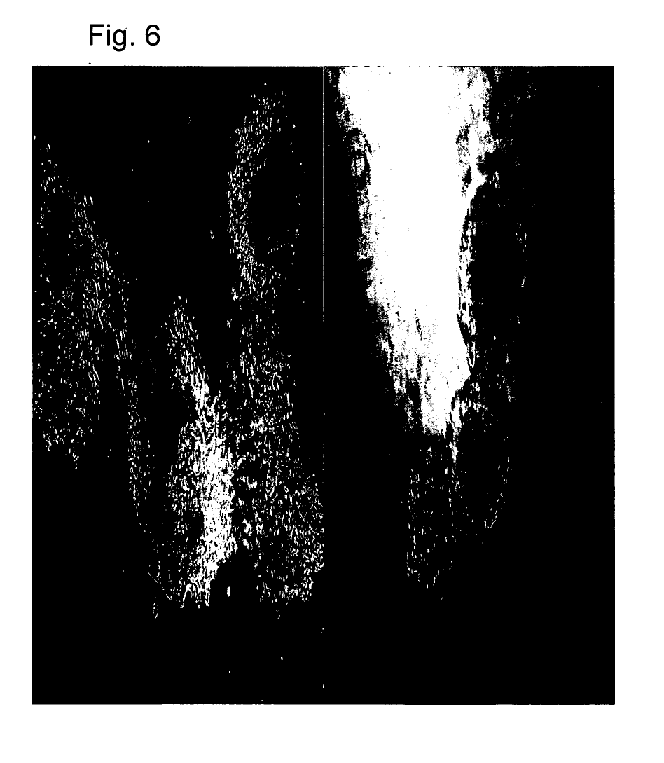

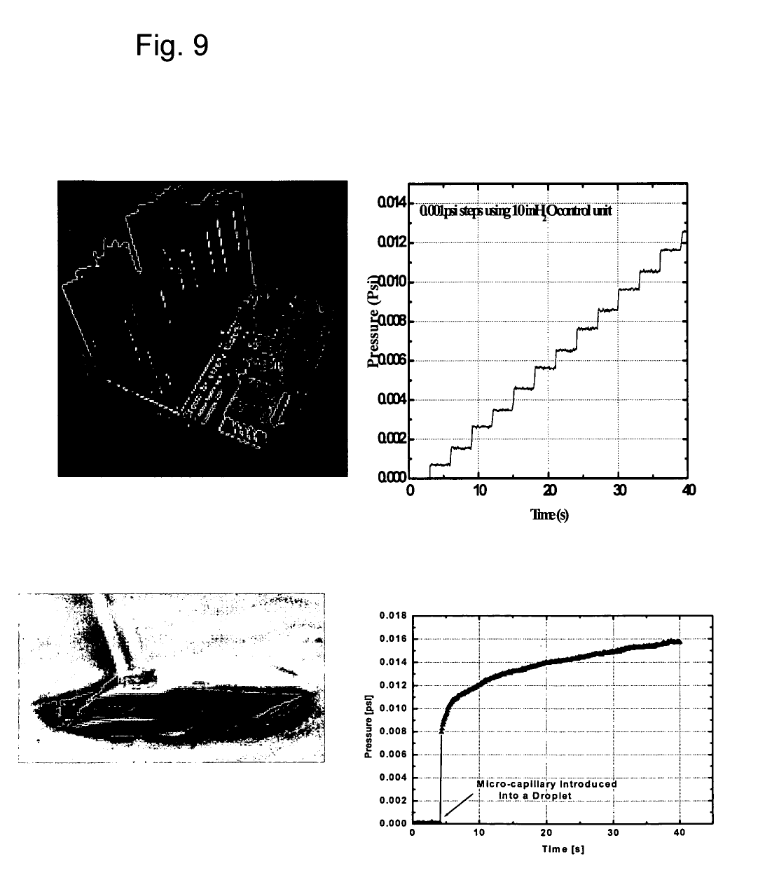

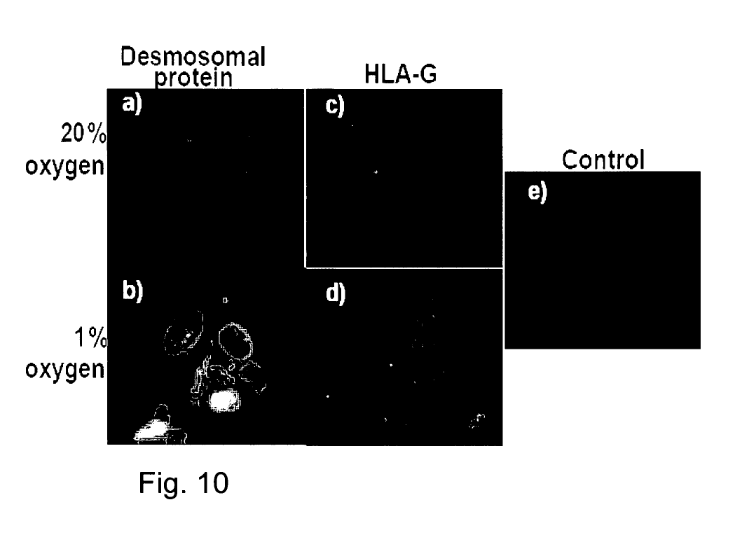

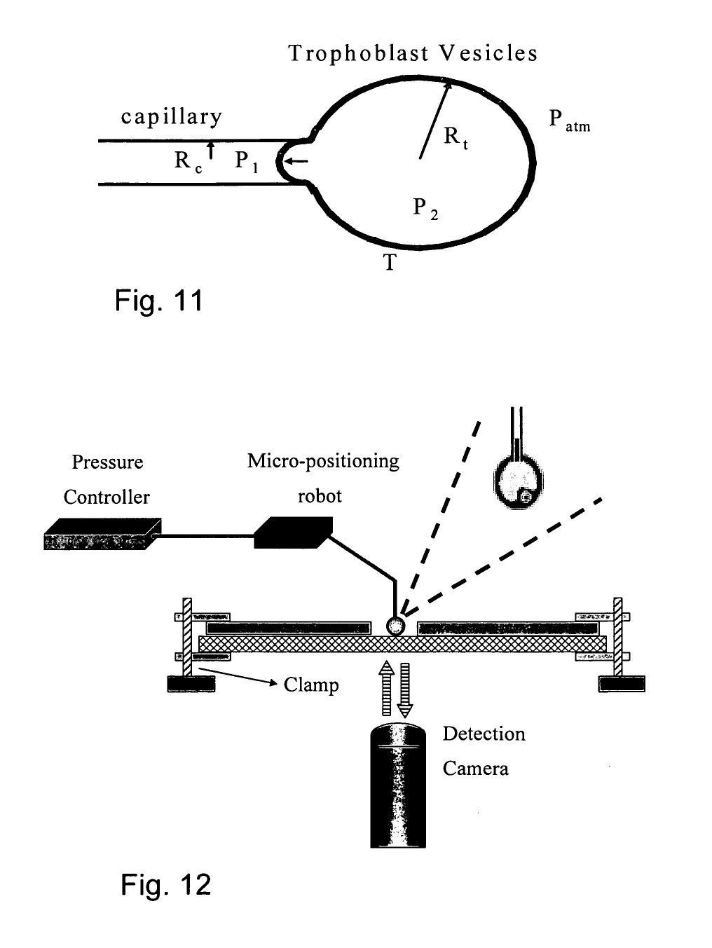

Referring now to FIG. 9, in order to measure/sense ultra-small (<5 inches of H₂O) pressures accurately with high signal to noise ratios (>100), a pressure probe is integrated into a microfluidic controller developed for imposing/measuring microfluidic pressures as small as 0.1 inches of water in micro channel networks.³³⁻³⁵ See also United States Patent Publication 2006/0193730 (U.S. Ser. No. 11/184,533 herein incorporated by reference herein in its entirety.) FIG. 6 shows a snapshot of the controller and the measured pressure inside a microfluidic channel with nanoliter flow. In this technique, a micro capillary filled with air is connected to a MEMS based pressure sensor with precision electrical circuitry. When the microcapillary is inserted inside a vesicle or droplet, the fluid rises to compresses the air. This results in compression of MEMS membrane. The pressure inside the vesicle or droplet can then be evaluated using the formula P_{vesicle}=P_{measured}−2γ cos θ/R. Here, the second term is a constant which quantifies the rise of fluid due to capillary action (R=radius of capillary, γ surface tension). This device, integrated with an accurate precision motion stage, may be employed for measuring pressure inside the de novo trophoblast vesicles of the invention. An alternative technique to indirectly calculate vesicle pressure is illustrated in FIG. 11.

### Example 5

### Cell Culture Environment Affect Trophoblast Cell Differentiation

Purified trophoblast cells were cultured in 21% and 2.5% oxygen tension for up to 5 days. RNA was obtained, reverse transcribed, and assayed by real-time PCR for markers of villous and extravillous trophoblast cell differentiation pathways. The cells grown in 21% oxygen increased the expression of hCG, hPL and syncytin, all markers of villous trophoblast differentiation. This is consistent with the known spontaneous differentiation of these cells in 21% oxygen and has been well characterized by several investigators. In contrast, cells grown in low oxygen demonstrated decreased expression of these villous markers and increased expression of HLA-G, a marker of extravillous trophoblast cells. Next, cells were cultured in 21% or 1% oxygen for five days and then stained for desmosomal protein or HLA-G. Desmosomal protein marks the cell membranes. By counterstaining the nuclei, the presence of multinucleated synctiotrophoblasts is identified. As anticipated, cells grown in ambient oxygen synctialized. Cells grown in 1% oxygen did not form a syncytium. Only the cells grown in low oxygen expressed HLA-G. Taken together, these data (genetic markers and immunohistochemistry) indicate that oxygen tension plays a role in determining the lineage of cytotrophoblast cell differentiation. The effect of oxygen tension on the differentiation and formation of trophoblast vesicles can be tested as described below.

### Example 6

### Testing Functional Capacity for Implantation

The de novo trophoblast vesicles have the functional capacity for implantation. Both in vitro and in vivo models of implantation can be created by those of skill in the art. Functional trophoblast vesicles will produce matrix metal proteinases and will implant into a basement membrane. This can be confirmed by demonstrating the production of MMP-2 and MMP-9 by RNA. The function of these proteins can be assessed by zymography. Implantation can be assessed by trophoblast invasion into basement membranes. Finally, the ability to implant in vivo can be assessed by transferring trophoblast vesicles to prepared mouse surrogates and calculating implantation rates nine days after transfer.

### Example 7

### Testing Pressure and Ionic Concentrations Inside Trophoblast Vesicles

To determine the relationship between trophoblast vesicle formation and increased spheroid pressure, it is necessary to have a description of the activities of its component and pressure in relation to time and environment. To our knowledge, there exists no data on the measurement of time dependent changes of pressure and ionic concentration in trophoblast vesicles. There have been many studies performed to measurement solute and pressure (turgor) in plant cells to understand metabolic functions.²⁹ Solute measurements can be addressed by the use of techniques such as ion-sensitive fluorescent probes,^{30, 31} that can be used to show that a particular component or compound is located in certain cell types. In some cases, however, quantitative precision of these techniques is doubtful. This is especially true where solutes are measured because these contribute to both metabolic and osmotic cell functions. In addition to measurements of cell turgor, the pressure measurements were also performed across red cells. The pressure across the red cell membrane has been estimated to be around 2 mm H₂O.³² No difference in stiffness was found between the rim and the biconcavity of the cell or between biconcave discs and hypotonically swollen cells. Above studies clearly suggest that changes in solute concentrations can have effects both on the rate of flux through biochemical pathways and on trophoblast vesicle pressure. Changes in the latter will have consequences on the functional capacity and growth of self-assembled three dimensional trophoblast vesicles.

### Example 8

### The Effect of Recess Geometry, Cell Number, and Oxygen Tension on Trophoblast Vesicle Morphology

Wax molds are created using computer assisted design and rapid prototyping with a variety of geometries. Configurations to be tested include cylindrical recess with diameters of 100, 200, 400, or 600 μm containing flat and cylindrical bottoms. These diameters are chosen because they approximate the size of mouse and human blastocysts. Non-cylindrical conical geometry can also be assessed. Trophoblast cells are seeded into the gels and cultured for up to 10 days. Cellular morphology is assessed with stereo and confocal microscopy and by fixing and sectioning the spheroids. Viability is assessed using live:dead assay. To determine the lineage of trophoblast cell differentiation, RNA is collected from the spheroids and the expression of villous (syncytin, hPL) and extravillous (HLA-G, αᵥβ₃) trophoblast cell markers is quantified with real-time PCR. Data is standardized to β-actin and 18S.

To determine the effect of cell number on vesicle morphology, trophoblast cells are seeded into the non-adhesive micro-mold cell aggregation devices at cell densities between 30 and 3000 cells per recess. Spheroids are allowed to form for 10 days. After 10 days, the spheroids are removed from the wells and the maximal dimensions (height and width) and morphology determined with confocal microscopy and serial sectioning. The live:dead assay is used to determine the percent of cellular viability within the vesicle. These experiments are repeated for each of the bioreactors created in the experiments above. In a parallel set of experiments, spheroids are formed for 10 days and RNA is collected to determine cellular patterns of differentiation into villous (syncytin, hPL) and extravillous (HLA-G, αᵥβ₃) trophoblast cell lineage pathways.

Because low oxygen tension promotes trophoblast cells in monolayer to differentiate along the extravillous trophoblast cell lineage pathway, low oxygen tension can induce extravillous trophoblast differentiation in trophoblast vesicles. Trophoblast cells are seeded into nonadhesive micro-mold bioreactors as determined by the two prior experiments. Cells are immediately placed in incubators containing 20% oxygen, 10% oxygen, or 2.5% oxygen. These oxygen tensions correspond to room air, oxygen tension in the intervillous space during the second trimester of pregnancy, and the oxygen tension in the intervillous space during early pregnancy.³⁶ The trophoblast vesicles are cultured for 10 days and then assessed for morphologic development and differentiation as noted above. Morphology is described with the use of confocal microscopy and serial sectioning. Differentiation is determined with real time PCR for villous and extravillous markers.

Cells are assessed for size with confocal microscopy. Horizontal (x, y plane) diameters of 10 aggregates per sample is measured by converting 3D image data into a 2D projection of average pixel intensity. These images are thresholded and analyzed for area (x, y plane) with Scion Image software. Assuming a circular geometry, diameters are calculated from the x, y area. Height in the z-dimension for 10 aggregates is determined by plotting image intensity profile (intensity as a function of the z-dimension). The height is calculated as the z distance over which an aggregate displayed intensities above a minimum threshold of 5%. The 3D reconstructions are obtained for qualitative assessment of aggregate shape. Comparisons among spheroids are made by comparing the mean areas, and two-dimensional diameters using ANOVA. Statistically significant F-ratios for the vitamin supplementation variable is followed up using Tukey's honestly significant difference technique for multiple comparisons.

The percentage of live cells is calculated by randomly selecting 10 high-powered fields, counting the number of total and live cells, and dividing the number live by the total. A minimum of 100 total cells are counted. Comparisons are made among the spheroids using Chi-square analysis.

Real-time PCR data from the markers of differentiation are evaluated for quality prior to data analysis. Data must have single peak melting curves at the appropriate Tm, amplification slopes must be parallel in the log view, amplification must occur subsequent to the 10^{th} cycle, there must be at least 5 points along the linear amplification, and the correlation coefficient must be greater the 0.990. Cycle thresholds (CT) are calculated using the PCR baseline subtraction curve fit option. Data can be input for analysis using the Q-gene software for real-time PCR. This software calculates cDNA quantity based on the efficiency of amplification of both the experimental and reference genes and allows the input of multiple reference genes. The quantity of cDNA is compared among the vesicles using ANOVA. Linear regression is used to identify an association between markers of differentiation and size and shape of the vesicles.

An optimal trophoblast vesicle will contain a perfect sphere with an acellular center. The cellular rim will contain viable cells with few dead cells. These cells will primarily differentiate along the extravillous trophoblast pathway.

The cell line that may be employed should express features of both villous and extravillous cells. Alternatively, primary trophoblast cells that can be obtained from term placenta using enzymatic dispersion may be employed. Another cell line that may be employed is the murine trophoblast stem cell line identified by Rossant.³⁷ These cells differentiate along the three trophoblast cell line pathways identified in the mouse.

### Example 9

### Mechanism of Trophoblast Vesicle Formation

The formation of de novo trophoblast vesicles was not predicted by previous studies using similar bioreactors or using hanging drop cultures. We hypothesize that the trophoblast cells are programmed to form vesicles. As the cells are seeded into the bioreactor, they settle into a cell clump by gravity. Once contact is made the cells reassemble into a spheroid. The resulting vesicle may be the de novo reassemble structure, may be created by cells in the center of the sphere undergoing apoptosis or necrosis, or may be formed by the accumulation of fluid.

To determine whether apoptosis or necrosis is responsible for the formation of trophoblast vesicles, trophoblast cells are seeded into the non-adhesive micro-molded cell aggregation devices described above. Each day, the vesicles are removed from the wells, fixed and serial sectioned to view progressive changes in morphology. Immunohistochemistry for caspase-3 and -7 are performed on these sections to identify presence of apoptosis. The serial sections are also viewed by transmission electron microscopy to further delineate between necrosis and apoptosis. To confirm the presence or absence of apoptosis, vesicles are collected daily from a parallel set of experiments, RNA is collected, and the expression of caspace-3 and -7 is measured by real-time PCR.

### Example 10

### The Trophoblast Cells Develop Cytoplasmic Membrane Na—K ATpase Pumps

Trophoblast cells are seeded into the cell aggregation device for up to ten days to form trophoblast vesicles. The vesicles are removed from the cell aggregation device, the cells are lysed, and plasma membranes are prepared from the homogenate. Protein resolution is performed with SDS-PAGE and transferred to nitrocellulose paper. Antibody blotting against the Na—K ATPase α-1 and \tilde{β}₁ subunits is performed to identify their presence. Other vesicles formed in the aggregation devices are measured for pressure using a standard intra-cytoplasmic sperm injection needle attached to a micro-positioning robot and pressure controller/sensor to measure the pressure in the center of the vesicle and in the media. The difference between the inside and outside of the vesicle is calculated. To demonstrate that vesicle pressure is due to Na—K ATPase activity, trophoblast vesicles are seeded in the nonadhesive micro-mold cell aggregation device. After 24 hours, the media is supplemented with and without ouabain, a cardiac glycoside Na—K pump inhibitor. The cells are cultured for 7 days and the morphology and intra-vesicle pressure assessed. To demonstrate that vesicle pressure is maintained by Na—K ATPase activity, trophoblast vesicles are formed as described. After 7 days, the media is supplemented with ouabain or vehicle. The morphology and intravesicle pressure is measured daily for 3 days.

Immunohistochemical staining is qualitatively assessed to demonstrate induction of apoptosis over time. These data can be confirmed by real time PCR data using the Qgene data analysis program as described above. The amount of Na—K ATPase is determined by Western blotting and chemiluminescence assay and the resulting x-ray bands are analyzed using ImageQuant software. For quantitative pressure data, the means, standard deviations and standard errors within groups are calculated using Microcal Origin (version 7.0).

To determine whether the vesicles will rupture upon insertion of the pressure probe, pressure measurements can be performed using the resistance to deformation or stiffness of the membranes of the trophoblast vesicles. Briefly, it requires a measure of the pressure and time required to draw a cell into a micropipette (see FIG. 8). The governing equations for this approach are:

$${P}_{2} - {P}_{1} = \frac{2 T}{{R}_{c}} ; {P}_{2} - {P}_{atm} = \frac{2 T}{{R}_{t}}$$

Trophoblast pressure and stiffness can be evaluated by measuring P₁, Rₜ.

### Example 11

### Trophoblast Vesicles Produce Matrix Metal-Proteineases and Invade into a Basement Membrane

De novo trophoblast vesicles are created as described above and cultured for up to 10 days. Standard media is changed ever 48 hours and the spent media is assayed via zymography for the presence of gelatinase. Migration assays and trophoblast outgrowth assays are performed to demonstrate the capacity for invasion. Briefly, the migration assay is performed by placing a fixed number of vesicles on a Matrigel coated Boyden chamber for 20 hours and counting the number of cells that pass through the membrane. Outgrowth assays are performed by culturing the vesicles on fibronectin coated plates for three days and calculating the area of outgrowth.

Normally developing blastocysts up-regulate receptors to fibronectin in a polarized fashion. This assay is employed identify the position and semi-quantify the concentration of fibronectin receptors on the bioengineered trophoblast vesicles. Briefly, the trophoblast vesicle is cultured in the presence of fibronectin coated fluorescent microspheres. After approximately one hour, the fluorescent intensity and position are calculated. The presence of fibronectin receptors indicates a functional trophoblast vesicle.

To establish the in vivo hormonal milieu necessary to support a pregnancy, pseudo-pregnant immunodeficient mouse surrogates are prepared by mating female mice with vasectomized males. Because the mouse is immuno-deficient, it will not reject the vesicle. (This model has been used to test several human tissues including endometriosis, transplanted into mice.^{38, 39}) Ten trophoblast vesicles are then transferred to a single uterine horn of each surrogate. Implantation should be detectable by embryonic day 9 (E.9). However, because the vesicles do not contain an inner cell mass, the pregnancy is not likely to continue beyond this point. Therefore, on E.9, the pregnant mice are euthanized, the uteri removed, and implantation rates calculated.

Data from zymography is captured using a fluorescent CCD camera and analyzed using ImageQuant software. The mean intensity decreased from background represents the amount of MMPs in the media. The migration assays are quantified by counting the number of vesicles that are stained on the membrane in the lower chamber of the transwell. The presence and position of the fibronectin receptors is qualitatively determined. If present to a significant degree, the amount of fluorescence can be calculated using ImageQuant software and compared among time points as described. The presence of implantation sites is identified. Implantation rates are calculated as the number of implantations divided by the total number of embryos transferred.

### Example 12

### Implantation of the Vesicles

Inner cell masses from different mice are injected into the trophoblast vesicles of the invention and the newly reconstructed blastocysts are transferred into prepared mouse surrogates following the methods of Rossant.^{23, 24} The trophoblast vesicles are implanted as follows. Female reproductive aged Nu/Nu mice (Charles River Laboratories) are mated with vasectomized C57B/6 males to prepare them for surrogacy. After successful mating as demonstrated by the presence of a copulation plug the females have ten trophoblast vesicles transferred to one uterine horn. Trophoblast vesicle transfer is performed intra-abdominally to mice under adequate anesthesia. Mice are anesthetized with an intra-peritoneal injection of 75 mg/kg of ketamine and 1 mg/kg of medetomidine to provide an adequate surgical of 20-30 minutes. A small incision is made in the animal's back, the uterus is identified, a puncture is made in the uterus and the embryos are transferred directly into the uterine horn. The incision is closed with 4.0 vicryl suture and the animals are closely observed until they have completely recovered from the anesthetic agent.

This technique can also be used for interspecific cloning following the methods of Polzin et al.²⁵

### Materials and Methods

Cell culture: TCL human trophoblast cells immortalized from third trimester chorion are maintained in RPMI containing 10% FBS, penicillin (100 U/ml), and streptomycin (100 μg/ml) in a water-jacketed incubator with a humidified atmosphere (5% CO₂/air) at 37° C. The cells are maintained in monolayer, the media is changed every 48 hours, and the cells are passaged when they reach 95% confluence for a maximum of 12 passages. Approximately 800,000 cells are suspended in 100 μl of media and added to the bioreactor. After thirty minutes at 37° C., 3 ml of media are added to wells containing the bioreactor.

Maintenance of oxygen tension: Oxygen is maintained in controlled atmosphere culture chambers (Biospherix, Redfield, N.Y.) that continuously monitor oxygen and CO₂ tension. Low oxygen is maintained by nitrogen displacement. To change media, manipulate the culture or to visualize the cells, the cells are placed in a glove box that controls oxygen, CO₂ and temperature. The glove box contains a microscope and CCD camera to view the cells and record changes.

Live:dead assay: Trophoblast cells are grown on coverslips placed in 12 well plates for up to 10 days. On days one through six, a coverslip IS removed and washed with 1000 volumes of PBS. The coverslips are then incubated with a solution containing 5 μM of carboxyfluorescein dye and 5 μM of propidium iodide at 37° C. for 45 minutes. The coverslips are inverted onto 10 μl of PBS placed on a microscopic slide and the number of red and green cells is calculated. The live cells will fluoresce green while the dead cells will fluoresce red. The percentage of live cells is calculated as the number of green fluorescent cells divided by the total number of fluorescent cells.

RNA Collection and Quantification: RNA is collected using RNA Stat 60 (Tel-Test, Friendswood, Tex.) per the manufacturer's instructions. For each condition, 2 μg of total RNA is reverse transcribed with SuperScript III (Invitrogen, Carlsbad, Calif.) as per the manufacturer's instructions to yield 10 ng/ul of cDNA. Each PCR reaction contains 10 ng cDNA, 10 μM of each primer, 1 μl of 2×Sybr Green, 8 μl of Hot Master Mix (Eppendorf, Westbury, N.Y.) and 7 μl DEPC-treated water. The PCR protocol includes 40 cycles of amplification and a melting curve is produced by a slow denaturate of the PCR products to validate the specificity of amplification. Small oligonucleotide primers are designed to span an intron and produce an amplicon of approximately 120 base pairs. All primers are tested for efficiency of amplification and only those primers with an efficiency of greater than 95% are utilized. The PCR results are confirmed for each primer by sequencing the final product after gel purification. The primers are:

| | Forward Primer | Reverse Primer |
|---|---|---|
| HLA-G | CCACAGATACCTGGAGAACG | TGGTGGCCTCATAGTC |
| β-HCG | TGTGCATCACCGTCAACAC | GGTAGTTGCACACCACCTGA |
| β-Actin | AGCACAGAGCCCTCGCCTTT | ACATGCCGGAGCCGTTGT |
| hPL | GCTATGCTCCAAGCCCATC | TGCAGGAATGAATACTTCTG |
| | | GTC |
| Syncytin | AGGTGGGTTTCCTGGGTTTGC | TGGTGTCAATGTTGTTGG |

Samples are normalized to β-actin and to 18s using Quantum RNA 18S internal standard (Ambion, Austin, Tex.) to account for the relative abundance of this RNA. Data is analyzed using the Q-gene real time PCR program that improves on the accuracy of 2(ΔCT) by taking into account the efficiency of amplification for both the experimental and standard genes.

Fixing, sectioning and staining cells for immunohistochemistry: Mold are inverted in the six-well dish and centrifuged at 300 rpm for 5 minutes. The medium containing the trophoblast vesicles is aspirated from each well and transferred into a 15 mL conical tube. The vesicles are then pelleted by centrifugation at 300 rpm for 5 minutes and the supernatant is discarded. Cell pellets are fixed with 4% paraformaldehyde for 10 minutes. The fixed pellet is resuspended in PBS and centrifuged at 300 rpm for 5 minutes. The fixative is removed and 1 drop of OCT is added to the pellet and the pellet tube is placed on dry ice at −80° C. for at least 30 minutes. The frozen pellet is placed in the cyrostat on a precooled cryomold with OCT as a base and allowed to freeze. It is then sectioned at 15 μm and collected onto precleaned slides.

For immunohistochemistry, the cells are blocked with 5% low fat milk for 30 minutes at room temperature. A monoclonal anti protein antibody (i.e. anti-caspace-3 and -9) is added to blocking solution at a 1:400 dilution and the cells are incubated at 37° C. for 30 minutes. Cells are subsequently washed with TBS-T and incubated for one hour with a 1:15 dilution of biotinylated anti-mouse immunoglobulin secondary antibody. This antibody is removed by washing with TBS-T followed by 0.3% H₂O₂ in distilled water. Following another wash with PBS, the cells are incubated with avidin biotin peroxidase complex diluted 1:15 in PBS followed by the peroxidase substrate AEC (3-amino-9-ethycarbazole) for about 5-15 minutes until color develops. The slide is washed and the cells are counterstained with hematoxylin.

Membrane preparations: Cells from the trophoblast vesicle are dispersed by gentle repetitive pipetting of the vesicle. The cells are lysed with freshly prepared ice-cold buffer containing Tris-HCl (50 mM), EDTA (5 mM), 1% Triton X-100, SDS (0.05%), NaF (50 mM), Na3VO4 (100 microM), B-glycerophosphate (10 mM), sodium pyrophosphate (10 mM), phospho-serine (1 mM), phospho-threonin (1 mM), phospho-tyrosine (1 mM), leupeptin (20 microg/ml), bacitracine (500 microg/ml), and PMFS (100 microg/ml) for 10 minutes. The cells are then centrifuged for 15 min at 4° C. at 13000×g and the supernatant is removed Bradford assay as described by the manufacturer.

Quantification of Na—K ATPase: 40 μg protein is separated by 6% SDS-PAGE under a reducing condition using 100 volts for 5 hours. The proteins are electrophoretically transferred from gels to nitrocellulose membranes. The membrane is blocked for 1 hour with 5% nonfat milk in TBS-T and then washed with TBS-T for 10 minutes×3. The membrane is incubated with a 1:100 concentration of a monoclonal antibody to Na—K ATPase α1 or β1 subunit (Abcam, Cambridge, Mass.) overnight at 4° C. The membranes is then washed and incubated for 1 hour with a 1:2000 dilution of horseradish peroxidase-conjugated goat anti-mouse antibody. The antigen-antibody complexes are detected with the ECL chemiluminescence detection kit (Amersham Biosciences, Piscataway, N.J.). Membranes are visualized by exposure to x-ray film, scanned, and quantified with the ImageQuant program. These experiments are performed at least three times. Each membrane is stripped and assayed for β-actin antibody to account for protein differences.

Measurement of vesicle pressure: A trophoblast measuring system is integrated to the pressure sensing/controller instrument illustrated in FIG. 6 as follows. The trophoblast pressure measuring system as illustrated in FIG. 9 is composed of a vesicle holding unit, an imaging unit, a pressure controller unit and a micropositioning unit. To minimize vibration, all units except the host computer will be mounted on a vibration isolation table. The cellular pressure sensor probe, connected to a readout circuit board, is mounted on a three degrees-of-freedom (DOF) micro robot, in which the axes each has a travel of 2.54 cm with a step resolution of 40 nm. A standard ICSI injection pipette (Cook K-MPIP-1000-5) tip section with a tip diameter of 5 um is attached on the probe tip. The imaging unit includes an inverted microscope, a charge-coupled device (CCD) camera, a peripheral component interconnect (PCI) frame grabber, and a host computer. A Nikon TE2000 inverted microscope is used with a 40 long working distance objective and a Hoffman modulation contrast condenser. The CCD camera is mounted on the side port of the microscope. The frame grabber captures 30 frames/s. The software unit samples the cell membrane deformation images and pressures synchronously. The membrane geometries in each frame of image are measured offline with a resolution of one pixel. This platform is also used to measure the effect of replacing Na+ and K+ on the evolution of trophoblast vesicles. The ionic strength is measured using fluorescence and electric potential measurements.

Na—K ATPase inhibition: Ouabain octahydrate (Sigma, St. Louis, Mo.) is prepared in water. Trophoblast vesicles are treated with ouabain at doses between 10 ng/ml to 10 ug/ml. Initial experiments can conducted to determine the maximal tolerated dose of the glycoside, but it is anticipated that this will be approximately 100 ng/ml. Media is supplemented with or without ouabain beginning 24 hours after seeding (to determine the role of Na—K ATPase on vesicle formation) or ten days after seeding (to determine the role of the ATPase in vesicle maintenance.

Zymography: The protein content of the media is measured using Bradford protein assay as described by the manufacturer. If necessary, protein may be concentrated with Vivaspin protein concentrators (Sartorius, Edgewood, N.Y.) per manufacturer's instructions. A 4% SDS-PAGE stacking gel is prepared with 40 mg of gelatin added to the lower buffer. Approximately 50 μg of protein is resolved using 100 volts for 5 hours. Once resolution is adequate, the gel is incubated in 0.5% Trition X-100 washing buffer for one hour followed by overnight incubation with digestion buffer (10 mM calcium chloride, 20 mM Tris acetic acid with pH of 7.5.) at 37° C. The gels are then stained with 0.1% coomasie brilliant blue in 13% acetic acid followed by 10% acetic acid and 5% methanol for 3 hours.

Migration assay: The upper well of a transwell is coated with Matrigel (1 mg/l) (Becton-Dickinson, Franklin Lakes, N.J.) and the lower well is filled with 1 ml of media containing 5 μg of fibronectin. Trophoblast vesicles are removed from the bioreactor with gentle centrifugation (300 rpm for 5 minutes), washed with RPMI containing 1% FBS, and placed on the Matrigel. The transwells are incubated for 24 hours at 37° C. The media in the lower chamber is then aspirated, the membrane is removed and stained with Diff-Quick (Fisher Scientific, Louisville, Ky.), and the number of invaded vesicles is counted.

Fibronectin Binding Assay: Green-fluorescent microspheres (Molecular Probes, Eugene, Oreg.) are prepared by incubating 200 μl of microspheres with 144 μg/ml of fibronectin-120 (FN-120.) Incubating the trophoblast vesicles with 50 mg/ml FN-120 for 1 hour augments the fibronectin binding capacity. Next, the vesicles are incubated with FN-120 coated microspheres for one hour, unbound spheres are removed, and the vesicles are fixed with 4% paraformaldehyde. The vesicles are viewed with an inverted microscope with fluorescent illumination and the positions of the maximal fluorescent intensity, identifying the majority of fibronectin receptors, are noted. To quantify the receptor, 10-12 regions of interest are identified across the area of maximal fluorescence and intensity is averaged using image analysis software.

Embryo transfer: Surrogate animals are prepared by mating a Nu/Nu female mouse with a vasectomized male (C57B1/6 mouse strain). The morning after the breeding pair has been mated, the presence of a copulatory plug is investigated. Animals that have been successfully mated will have the trophoblast vesicle transfer performed. A pulled Pasteur pipette with a flame-polished tip is loaded with oil, a small amount of air, and then ten vesicles are loaded in approximately 50 μl of media. The recipient female is anesthetized with an intra-peritoneal injection of 75 mg/kg of ketamine and 1 mg/kg of medetomidine to provide an adequate surgical of 20-30 minutes. The lower back is then shaved and cleaned with 70% ethanol. A 1 cm incision is made in the abdomen and the fat pad and ovary are identified. The ovary is retracted and the infindibulum of the oviduct is located. The pipette is carefully placed into the oviduct and the ten vesicles are transferred. The oviduct is replaced in the abdomen and the incision is closed with 4.0 vicryl suture and a skin staple. The mouse is monitored frequently until she completely recovers from anesthesia.

### CONCLUSION

The de novo formation of anembryonic trophoblast vesicles as described above enables scientists to study the complex events involved with blastocyst formation, implantation, and placental development, events that can not be adequately modeled with currently available models. These studies will increase our knowledge about the etiologies of infertility and diseases of placentation including preeclampsia. The method and compositions of the invention can be employed to test mechanisms of implantation and to develop culture methods to improve implantation.

The compositions and methods disclosed and herein can be made and executed without undue experimentation in light of this disclosure. Although the compositions and methods of the invention have been described in terms of preferred embodiments, it will be apparent to those having ordinary skill in the art that variations may be made to the compositions and methods without departing from the concept, spirit and scope of the invention. Fore example, certain agents and compositions that are chemically related may be substituted for the agents and compositions described herein if the same or similar results would be achieved. All such similar substitutes and modifications apparent to those skilled in the art are deemed to be within the spirit, scope, and concept of the invention. All publications, patent applications, patents and other documents cited herein are incorporated by reference in their entirety. In case of conflict, this specification including definitions will control. In addition, the material, methods, and examples are illustrative only and not intended to be limiting.

### REFERENCES

1. Brewer J L. A normal human ovum in a stage preceding the primitive streak. Am J Anat. 1937; 61:429-481.
2. Morrish D W, Dakour J, Li H. Functional regulation of human trophoblast differentiation. J Reprod Immunol. August 1998; 39(1-2):179-195.
3. Conley A J, Mason J I. Placental steroid hormones. Baillieres Clin Endocrinol Metab. June 1990; 4(2):249-272.
4. Lyall F. Mechanisms regulating cytotrophoblast invasion in normal pregnancy and pre-eclampsia. Aust N Z J Obstet. Gynaecol. August 2006; 46(4):266-273.
5. Norwitz E R. Defective implantation and placentation: laying the blueprint for pregnancy complications. Reprod Biomed Online. October 2006; 13(4):591-599.
6. Mi S, Lee X, Li X, et al. Syncytin is a captive retroviral envelope protein involved in human placental morphogenesis. Nature. Feb. 17, 2000; 403(6771):785-789.
7. Rama S, Rao A J. Regulation of growth and function of the human placenta. Mol Cell Biochem. November 2003; 253(1-2):263-268.
8. Rama S, Suresh Y, Rao A J. TGF beta1 induces multiple independent signals to regulate human trophoblastic differentiation: mechanistic insights. Mol Cell Endocrinol. Aug. 29, 2003; 206(1-2):123-136.
9. Robins J C, Heizer A, Hardiman A, Hubert M, Handwerger S. Oxygen tensions direct the differentiation pathway of human cytotrophoblast cells. Placentta. 2007;In press.
10. Dozmorov M G, Kyker K D, Saban R, et al. Analysis of the interaction of extracellular matrix and phenotype of bladder cancer cells. BMC Cancer. 2006; 6:12.
11. Martin I, Wendt D, Heberer M. The role of bioreactors in tissue engineering. Trends Biotechnol. February 2004; 22(2):80-86.
12. Vunjak-Novakovic G. The fundamentals of tissue engineering: scaffolds and bioreactors. Novartis Found Symp. 2003; 249:34-46; discussion 46-51, 170-174, 239-141.
13. Gordon J N, Shu W P, Schlussel R N, Droller M J, Liu B C. Altered extracellular matrices influence cellular processes and nuclear matrix organizations of overlying human bladder urothelial cells. Cancer Res. Oct. 15, 1993; 53(20):4971-4977.
14. Wendt D, Stroebel S, Jakob M, John G T, Martin I. Uniform tissues engineered by seeding and culturing cells in 3D scaffolds under perfusion at defined oxygen tensions. Biorheology. 2006; 43(3-4):481-488.
15. Fassina L, Saino E, Visai L, De Angelis M G, Benazzo F, Magenes G. Enhanced in vitro culture of human SAOS-2 osteoblasts on a sand-blasted titanium surface modified with plastic deformation. Conf Proc IEEE Eng Med Biol Soc. 2007; 1:6410-6413.
16. Timmins N E, Harding F J, Smart C, Brown M A, Nielsen L K. Method for the generation and cultivation of functional three-dimensional mammary constructs without exogenous extracellular matrix. Cell Tissue Res. April 2005; 320(1):207-210.
17. Dean D M, Napolitano A P, Youssef J, Morgan J R. Rods, tori, and honeycombs: the directed self-assembly of microtissues with prescribed microscale geometries. Faseb J. December 2007; 21(14):4005-4012.
18. Napolitano A P, Chai P, Dean D M, Morgan J R. Dynamics of the self-assembly of complex cellular aggregates on micromolded nonadhesive hydrogels. Tissue Eng. August 2007; 13(8):2087-2094.
19. Napolitano A P, Dean D M, Man A J, et al. Scaffold-free three-dimensional cell culture utilizing micromolded nonadhesive hydrogels. Biotechniques. October 2007; 43(4):494, 496-500.
20. Claude P. Morphological factors influencing transepithelial permeability: a model for the resistance of the zonula occludens. J Membr Biol. Mar. 10, 1978; 39(2-3):219-232.
21. Sheth B, Fesenko I, Collins J E, et al. Tight junction assembly during mouse blastocyst formation is regulated by late expression of ZO-1 alpha+isoform. Development. May 1997; 124(10):2027-2037.
22. Madan P, Rose K, Watson A J. Na/K-ATPase beta1 subunit expression is required for blastocyst formation and normal assembly of trophectoderm tight junction-associated proteins. J Biol. Chem. Apr. 20, 2007; 282(16):12127-12134.
23. Rossant J, Lis W T. Potential of isolated mouse inner cell masses to form trophectoderm derivatives in vivo. Dev Biol. May 1979; 70(1):255-261.
24. Rossant J, Mauro V M, Croy B A. Importance of trophoblast genotype for survival of interspecific murine chimaeras. J Embryol Exp Morphol. June 1982; 69:141-149.
25. Polzin V J, Anderson D L, Anderson G B, et al. Production of sheep-goat chimeras by inner cell mass transplantation. J Anim Sci. July 1987; 65(1):325-330.
26. Kunath T, Arnaud D, Uy G D, et al. Imprinted X-inactivation in extra-embryonic endoderm cell lines from mouse blastocysts. Development. April 2005; 132(7):1649-1661.
27. Tanaka S, Kunath T, Hadjantonakis A K, Nagy A, Rossant J. Promotion of trophoblast stem cell proliferation by FGF4. Science. Dec. 11, 1998; 282(5396):2072-2075.
28. Pfeifer A, Ikawa M, Dayn Y, Verma I M. Transgenesis by lentiviral vectors: lack of gene silencing in mammalian embryonic stem cells and preimplantation embryos. Proc Natl Acad Sci USA. Feb. 19, 2002; 99(4):2140-2145.
29. Tomos A D, Leigh R A. The pressure probe: A versatile tool in plant cell physiology. Annual Review of Plant Physiology and Plant Molecular Biology. 1999; 50:447-+.
30. Bouchelouche P N. Dynamic, real time imaging of ion activities in single living cells using fluorescence video microscopy and image analysis. Scand J Clin Lab Invest Suppl. 1993; 214:27-39.
31. Muallem S, Zhang B X, Loessberg P A, Star R S. Simultaneous recording of cell volume changes and intracellular pH or Ca2+ concentration in single osteosarcoma cells UMR-106-01. J Biol. Chem. Sep. 5, 1992; 267(25):17658-17664.
32. Rand R P, Burton A C. Mechanical Properties of the Red Cell Membrane. I. Membrane Stiffness and Intracellular Pressure. Biophys J. March 1964; 4:115-135.
33. Kerby M B, Lee J, Ziperstein J, Tripathi A. Kinetic measurements of protein conformation in a microchip. Biotechnol Prog. September-October 2006; 22(5):1416-1425.
34. Kerby M B, Legge R S, Tripathi A. Measurements of kinetic parameters in a microfluidic reactor. Anal Chem. Dec. 15, 2006; 78(24):8273-8280.
35. Lee J, Tripathi A. Intrinsic viscosity of polymers and biopolymers measured by microchip. Anal Chem. Nov. 15, 2005; 77(22):7137-7147.
36. Jauniaux E, Watson A L, Hempstock J, Bao Y P, Skepper J N, Burton G J. Onset of maternal arterial blood flow and placental oxidative stress. A possible factor in human early pregnancy failure. Am J. Pathol. December 2000; 157(6):2111-2122.
37. Rossant J. Stem cells and lineage development in the mammalian blastocyst. Reprod Fertil Dev. 2007; 19(1):111-118.
38. Fortin M, Lepine M, Merlen Y, et al. Quantitative assessment of human endometriotic tissue maintenance and regression in a noninvasive mouse model of endometriosis. Mol. Ther. April 2004; 9(4):540-547.
39. Grummer R. Animal models in endometriosis research. Hum Reprod Update. September-October 2006; 12(5):641-649.

1. Anembryonic, de novo, trophoblast vesicles further characterized by (a) having the functional capacity for implantation, (b) being composed of a substantially perfect sphere having a hollow, acellular center, (c) having a cellular rim containing viable cells that are proliferating, and (d) having numerous tight junctions among the viable cells in the rim.

2. Anembryonic, de novo, trophoblast vesicles according to claim 1 wherein the functional capacity for implantation is exhibited by production of MMP-2 and MMP-9.

3. Anembryonic, de novo, trophoblast vesicles according to claim 1 wherein the vesicles are formed from trophoblast cells seeded into a cell aggregation device.

4. Anembryonic, de novo, trophoblast vesicles according to claim 1 wherein the vesicles demonstrate Na—K ATPase activity.

5. Anembryonic, de novo, trophoblast vesicles according to claim 1 wherein the vesicles implant when transferred into a uterine horn of a pseudo-pregnant, immunodeficient mouse surrogate.

6. A method of making anembryonic de novo trophoblast vesicles comprising seeding trophoblast cells in a non-adherent cell aggregation device and incubating the cells in the device until the cells form anembryonic trophoblast vesicles.

7. The method of claim 6 wherein the cell aggregation device is composed of agarose.

8. The method of claim 7 wherein the cell aggregation device is formed having a plurality of cell-repellant compartments recessed into the uppermost surface wherein each compartment is composed of an upper cell suspension seeding chamber having an open uppermost portion and bottom portion, and a lower cell aggregation recess connected at the top to the bottom of the upper cell suspension seeding chamber by a port.

9. Anembryonic, de novo, trophoblast vesicles made by the method according to claim 6.


SEQUENCE LISTING (10 sequences)

SEQ ID NO: 1 - DNA, 20 bp, Artificial Sequence
  HLA-G Forward Primer
ccacagatacctggagaacg

SEQ ID NO: 2 - DNA, 16 bp, Artificial Sequence
  HLA-G Reverse Primer
tggtggcctcatagtc

SEQ ID NO: 3 - DNA, 19 bp, Artificial Sequence
  Beta-HCG Forward Primer
tgtgcatcaccgtcaacac

SEQ ID NO: 4 - DNA, 20 bp, Artificial Sequence
  Beta-HCG Reverse Primer
ggtagttgcacaccacctga

SEQ ID NO: 5 - DNA, 20 bp, Artificial Sequence
  Beta-Actin Forward Primer
agcacagagccctcgccttt

SEQ ID NO: 6 - DNA, 18 bp, Artificial Sequence
  Beta-Actin Reverse Primer
acatgccggagccgttgt

SEQ ID NO: 7 - DNA, 19 bp, Artificial Sequence
  hPL Forward Primer
gctatgctccaagcccatc

SEQ ID NO: 8 - DNA, 23 bp, Artificial Sequence
  hPL Reverse Primer
tgcaggaatgaatacttctggtc

SEQ ID NO: 9 - DNA, 21 bp, Artificial Sequence
  Syncytin Forward Primer
aggtgggtttcctgggtttgc

SEQ ID NO: 10 - DNA, 18 bp, Artificial Sequence
  Syncytin Reverse Primer
tggtgtcaatgttgttgg